(19) United States
(12) Patent Application Publication
(10) Pub. No.: US 2025/0356065 A1
(43) Pub. Date: Nov. 20, 2025

(54) METHOD MAKING IT POSSIBLE TO PRODUCE THE IDEAL CURVATURE OF A ROD OF VERTEBRAL OSTEOSYNTHESIS MATERIAL DESIGNED TO SUPPORT A PATIENT'S VERTEBRAL COLUMN

(71) Applicant: Medicrea International, Rillieux-La-Pape (FR)

(72) Inventors: Thomas Mosnier, Rochetaillée Sur Saône (FR); David Ryan, Rochetaillée Sur Saône (FR); Vincent Fiere, Lyon (FR)

(73) Assignee: Medicrea International, Rillieux-La-Pape (FR)

(21) Appl. No.: 19/281,469

(22) Filed: Jul. 25, 2025

Related U.S. Application Data
(63) Continuation of application No. 18/752,014, filed on Jun. 24, 2024, now Pat. No. 12,417,323.
Application No. 18/752,014 is a continuation of application No. 17/188,261, filed on Mar. 1, 2021, now Pat. No. 12,019,955.
Application No. 17/188,261 is a continuation of application No. 16/389,348, filed on Apr. 19, 2019, now Pat. No. 10,970,426.
Application No. 16/389,348 is a continuation of application No. 14/914,474, filed on Feb. 25, 2016, now Pat. No. 10,318,655.

(30) Foreign Application Priority Data
Sep. 18, 2013 (FR) 1358988

(51) Int. Cl. G06F 30/00; A61B 17/56; A61B 17/70; A61B 34/10

(52) U.S. Cl. CPC G06F 30/00; A61B 17/7011; A61B 34/10; A61B 2017/568; A61B 2034/105; A61B 2034/108

(57) ABSTRACT

A method and system for producing a curvature of a rod of vertebral osteosynthesis material designed to support a vertebral column of a patient is provided. A production unit receives a model defining the curvature of the rod. The curvature of the rod is based on a preoperative X-ray of the vertical column and defined by a simulated correction to be applied to the lumbar segment to be treated, a deduced curved segment representing the curvature of the rod based at least in part on one or more of a lumbar lordosis or a first curvature above a repositioned apex point and the second curvature below the repositioned apex point, and a translated curved segment representative of the deduced curved segment being translated away from a mean line of the vertebral column. The production unit uses computer aided design to produce the rod.

### CROSS-REFERENCE TO RELATED APPLICATION

This application is a continuation of U.S. application Ser. No. 18/752,014, filed Jun. 24, 2024, which is a continuation of U.S. application Ser. No. 17/188,261, filed Mar. 1, 2021, which is a continuation of U.S. application Ser. No. 16/389,348, filed Apr. 19, 2019, now U.S. Pat. No. 10,970,426, which is a continuation of U.S. application Ser. No. 14/914,474, filed Feb. 25, 2016, now U.S. Pat. No. 10,318,655, which is a national stage entry of PCT/IB2014/064586 filed Sep. 17, 2014, under the International Convention claiming priority over French Patent Application No. 1358988 filed Sep. 18, 2013, all of which are incorporated herein by reference in their entirety.

### FIELD OF THE INVENTION

The present invention relates to a method making it possible to produce the ideal curvature of a rod of vertebral osteosynthesis material designed to support a patient's vertebral column.

### BACKGROUND OF THE INVENTION

It is known to analyze a patient's vertebral column in reference to so-called “pelvic” parameters and different morphotypes of a vertebral column, documented in the scientific literature. The appended FIG. 1 very diagrammatically shows the base of the vertebral column, i.e., part of the lumbar vertebrae Land the sacrum S, as well as the femoral heads TF; the aforementioned pelvic criteria are:
the SS (sacral slope) criterion, which is the incline angle of the plate of S1 (first vertebra of the sacrum), relative to the horizontal;
the PV (pelvic version) criterion, which is the angle formed by the straight segment connecting the center of the femoral heads TF and the center of the plate of S1 with the vertical;
the Pl (pelvic incidence) criterion, which is the angle formed by the straight segment connecting the center of the femoral heads TF and the center of the plate of S1 with the perpendicular to the plate of S1.

FIGS. 2A to 2D respectively show:
a so-called “type 1” morphotype, in which the apex (i.e., the forwardmost point of the vertebral column) is situated at the median plane of L5 (fifth lumbar vertebra), and the SS criterion corresponds to an angle smaller than 35°;
a so-called “type 2” morphotype, in which the apex is situated at the base of L4 (fourth lumbar vertebra), and the SS criterion corresponds to an angle smaller than 35°;
a so-called “type 3” morphotype, in which the apex is situated at the median plane of LA, and the SS criterion corresponds to an angle comprised between 35° and 45°;
a so-called “type 4” morphotype, in which the apex is situated at the base of L3 (third lumbar vertebra), and the SS criterion corresponds to an angle larger than 45°.

It is accepted that an individual will adopt a natural vertebral column posture, called “economic”, avoiding pain and other pathologies, if his pelvic parameters in particular are in agreement with his back morphotype. If this is not the case, surgical treatment may be considered in order to reestablish proper posture of the vertebral column, in which that agreement exists.

It is well known to perform this type of recovery using rigid vertebral rods, in particular made of metal, fastened to the vertebrae using anchor members such as pedicle screws or laminar hooks, which rods must be curved suitably based on the correction to be done. The publication of patent application No. WO 98/55038 illustrates material of this type.

It has been shown that imparting the appropriate curvature to a straight rod may be very difficult for a surgeon, the curvature being more or less pronounced in any given location of the rod. Currently, such a curvature is done at the surgeon's discretion and calls greatly on the latter's experience and dexterity. The trial and error necessary to obtain an appropriate curvature have the significant drawback of extending the operation time, which is not desirable for the patient, and the risk of implanting a rod with a non-ideal curvature cannot be ruled out.

### OBJECTS OF THE INVENTION

The present invention aims to resolve this essential drawback.

The patent application publications No. WO 2004/017836 A2, WO 2009/254326 A 1 and US 2008/079546 A2 describe methods that do not achieve this goal satisfactorily.

### SUMMARY OF THE INVENTION

To that end, the method according to the invention comprises the following steps:
a) taking a sagittal preoperative x-ray of the vertebral column of the patient to be treated, extending from the cervical vertebrae to the femoral heads;
b) on that x-ray, identifying:
   the so-called “pelvic” parameters, i.e., the sacral slope, the pelvic version and the pelvic incidence,
   the lumbar lordosis,
   the position of the apical lumbar vertebra, i.e., that situated furthest forward on the x-ray,
   at least one of the following measurements:
      the distance, called SVA, from the vertical of the posterior upper point of the plate of the first vertebra of the sacrum, called S1, to the vertical passing through the center of the 7th cervical vertebra;
      the distance, called SFD, from the vertical of the posterior upper point of the plate of S1 to the vertical passing through the center of the femoral heads;
      the angle, called T1/SPI, formed between the segment going from the center of the first dorsal vertebra, called T1, to the center of the femoral heads and the vertical to the center of T1, a cloud of points defining the curvature of the patient's vertebral column, including one point per vertebral level, positioned at the center of the upper plate of an affected vertebra, and a point defining the noted preoperative apex;
c) deducing, among predetermined vertebral column morphotypes and from the noted value of the sacral slope, the morphotype to which the treated vertebral column corresponds and deducing the desired postoperative apex point after performing the correction therefrom, and defining the vertebrae in which the anchor members for the vertebral rod to be anchored to the vertebrae will be implanted;
d) performing a wire modeling of the patient's vertebral column, for example using CAD software;
e) defining, from said pelvic parameters, a reference centered at the plate of S1, the origin point of which is the central point of that plate;
f) positioning the different points of said cloud of points, attached to each vertebra, in that reference, and drawing arcs step by step between the identified points, all of the arcs being tangent to each other and the arc extending from S1 being tangent to the straight line perpendicular to the plate of S1;
g) reading the values of the arc lengths;
h) simulating the correction to be applied to the lumbar segment to be treated as follows:
   h1) drawing a straight line tangent to the desired postoperative apex point, moving that straight line to a vertical position such that the arc attached to that straight line is tangent to the desired postoperative apex point, the latter thus being repositioned so as to become the apex point of the modeled vertebral column segment,
   h2) defining, as co-radial to each other, the arcs situated below that apex point and defining as co-radial to each other the arcs situated above that same apex point, so as to obtain two different curvatures, one above that apex point and the other below the apex point,
   h3) defining the lumbar lordosis as being equal to plus or minus ten degrees of the pelvic incidence, and defining one of the following three values as desired:
      SVA distance smaller than 5 cm;
      SVA/SFD ratio comprised between −1.9 and +0.1;
      the value of this ratio is positive on the side of the vertical of the posterior upper point of the plate of S1 situated toward the femoral heads and is negative on the side of the vertical situated opposite the femoral heads;
      T1/SPI angle comprised between −9° and 0°, that angle being negative on the side of the vertical at the center of T1 located toward the femoral heads;
   h4) defining two arcs concentric to the two curvatures obtained during step h2 above, which are tangent to each other at the apex point, those arcs forming a curved segment representing the ideal curvature of the rod to be implanted in order to obtain the correction of the vertebral segment to be treated,
   h5) translating that curved segment away from the mean line of the vertebral column, over an evaluated mean distance going from the center of the vertebrae to the anchor points of the anchor members for anchoring the rod to the vertebrae of said vertebral segment to be treated, such that the position of said curved segment corresponds to the position the rod will assume once implanted;
   i) defining the diameter of the rod to be implanted;
   j) defining a two- or three-dimensional model of the rod, curved along said curved segment, and
   k) from a straight rod, producing the curvature of that rod according to said model.

Preferably, said predetermined vertebral column morphotypes comprise:
a so-called “type 1” morphotype, in which the apex (i.e., the forwardmost point of the vertebral column) is situated at the median plane of L5 (fifth lumbar vertebra), and the SS criterion corresponds to an angle smaller than 35°;
a so-called “type 2” morphotype, in which the apex is situated at the base of L4 (fourth lumbar vertebra), and the SS criterion corresponds to an angle smaller than 35°;
a so-called “type 3” morphotype, in which the apex is situated at the median plane of LA, and the SS criterion corresponds to an angle comprised between 35° and 45°;
a so-called “type 4” morphotype, in which the apex is situated at the base of L3 (third lumbar vertebra), and the SS criterion corresponds to an angle larger than 45°.

Preferably, the two- or three-dimensional modeling done in step j) consists of establishing a drawing or a plan of the rod to be produced.

Preferably, the curvature produced in step k) is done by cold bending.

Preferably, the method comprises, after step h5) or step i) or step j), the transfer of data relative to the rod to be produced to a service provider responsible for producing the curvature of the rod.

Thus, a practitioner, having determined the shape of the rod to be implanted using the method according to the invention, transfers the data relative to the rod to be produced to a service provider responsible for producing the curvature of the rod. Once that curvature is produced, the service provider will deliver the curved rod to the practitioner, who will be able to operate on the patient with his vertebral rod that is ready to be implanted.

The invention will be well understood, and other features and advantages thereof will appear, in reference to the appended diagrammatic drawing, showing, as a non-limiting example, different values used to carry out the method in question and different operations performed in the context of that implementation.

### BRIEF DESCRIPTION OF THE DRAWINGS

FIG. 1 very diagrammatically shows the base of the vertebral column;

FIG. 2A shows a so-called “type 1” morphotype, in which the apex is situated at the median plane of L5 and the SS criterion corresponds to an angle smaller than 35°;

FIG. 2B shows a so-called “type 2” morphotype, in which the apex is situated at the base of L4 and the SS criterion corresponds to angle smaller than 35°;

FIG. 2C shows a so-called “type 3” morphotype, in which the apex is situated at the median plane of LA and the SS criterion corresponds to angle comprised between 35° and 45°;

FIG. 2D shows a so-called “type 4” morphotype, in which the apex is situated at the base of L3 and the SS criterion corresponds to angle larger than 45°;

FIG. 3 is a very diagrammatic view of a vertebral column, on which the definition points of an SVA value used to implement the method according to the invention are shown;

FIG. 4 is a view similar to FIG. 3, which shows the definition points of the SVA value and an SFD value, those two values being used to define a ratio employed to carry out the method according to the invention;

FIG. 5 is a view similar to FIG. 3, showing the definition points of a so-called T1/SPI value used to carry out the method according to the invention;

FIGS. 6 to 11 are very diagrammatic views of reference points, segments, arcs of circle and curves used during the different successive steps of this method; and

FIG. 12 is, on the left side of that figure, a view of a drawing or plan P of the curved vertebral rod to be obtained, the shape of which has been defined by the preceding steps of the method and, on the right side of that figure, a view of the curved vertebral rod TV, obtained from that drawing P.

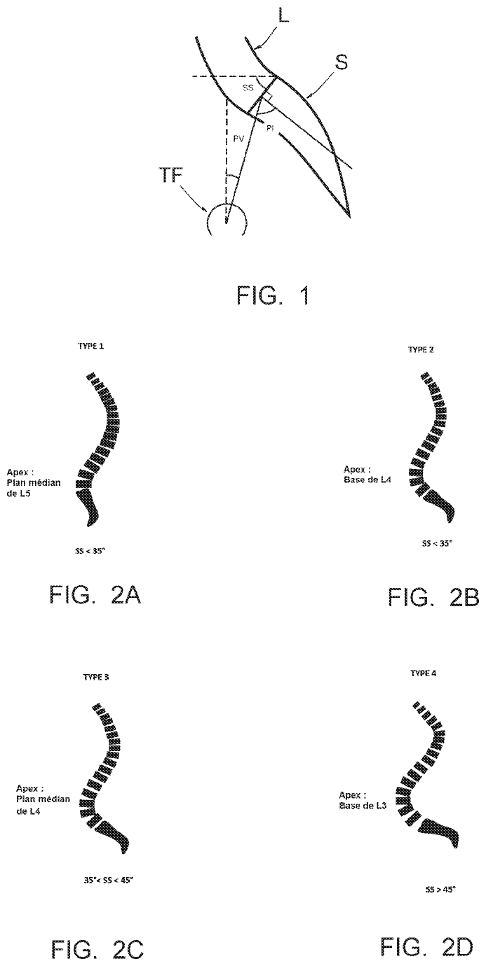

### DETAILED DESCRIPTION OF THE PREFERRED EMBODIMENT

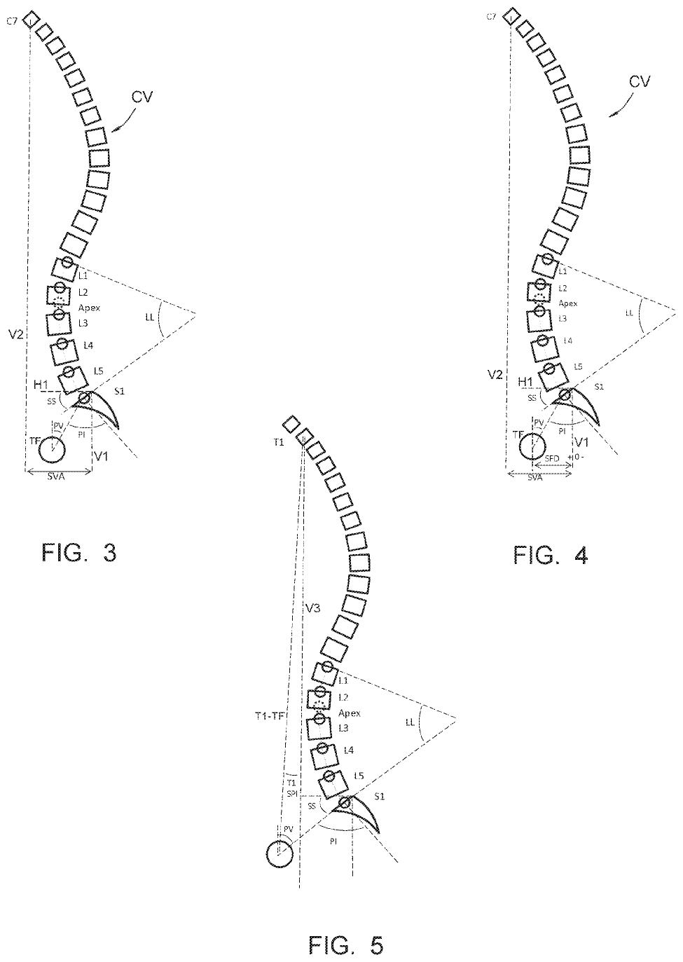

FIG. 3 very diagrammatically shows a vertebral column CV, and includes the following information:
LL: vertebral segment to be treated;
L1, L2, L3, L4, L5, S1, C7: the first, second, third, fourth and fifth lumbar vertebrae, the first vertebra of the sacrum and the seventh cervical vertebra, respectively;
Apex: the forward most point of the vertebral column; SS, PV, Pl: the aforementioned pelvic criteria;
TF: the femoral heads, shown by a circle;
H1 and V1: the horizontal and vertical, respectively, at the posterior upper point of the plate of S1;
V2: the vertical passing through the center of the 7th cervical vertebra (C7).

FIG. 4 also mentions a so-called “SFD” value, going from the vertical V1 to the vertical passing through the center of the femoral heads TF.

FIG. 5 also mentions a so-called “T1/SPI” value, which is the angle formed between the T1-TF segment going from the center of the first dorsal vertebra, called T1, to the center of the femoral heads TF and the vertical V3 to the center of T1.

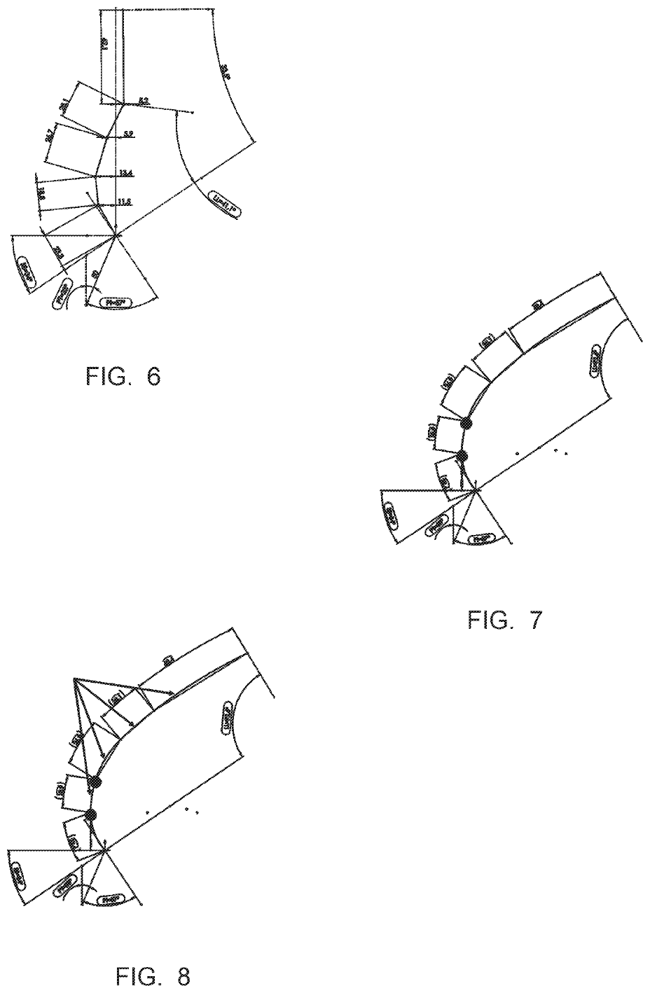

The various steps of the method are illustrated in FIGS. 6 to 11 as follows.

FIG. 6: on a sagittal preoperative x-ray of the vertebral column of the patient to be treated, extending from the cervical vertebrae to the femoral heads, the following are identified:
the pelvic parameters,
the lumbar lordosis, the position of the apical lumbar vertebra, i.e., that situated furthest forward on the x-ray,
as desired, the SVA distance only, or the SVA distance and the SFD distance,
a cloud of points defining the curvature of the patient's vertebral column, including one point per vertebral level, positioned at the center of the upper plate of an affected vertebra, and a point defining the noted preoperative apex.

The morphotype to which the treated vertebral column corresponds is deduced on the noted points, among the vertebral column morphotypes shown in FIGS. 2A to 2D, from the value of the noted sacral slope; the desired postoperative apex point after performing the correction is deduced therefrom; the vertebrae in which the anchoring members for the vertebral rod to be anchored to the vertebrae will be implanted are also defined in this step.

FIGS. 7 and 8: a wire model of the vertebral column of the patient is done, for example using CAD software;

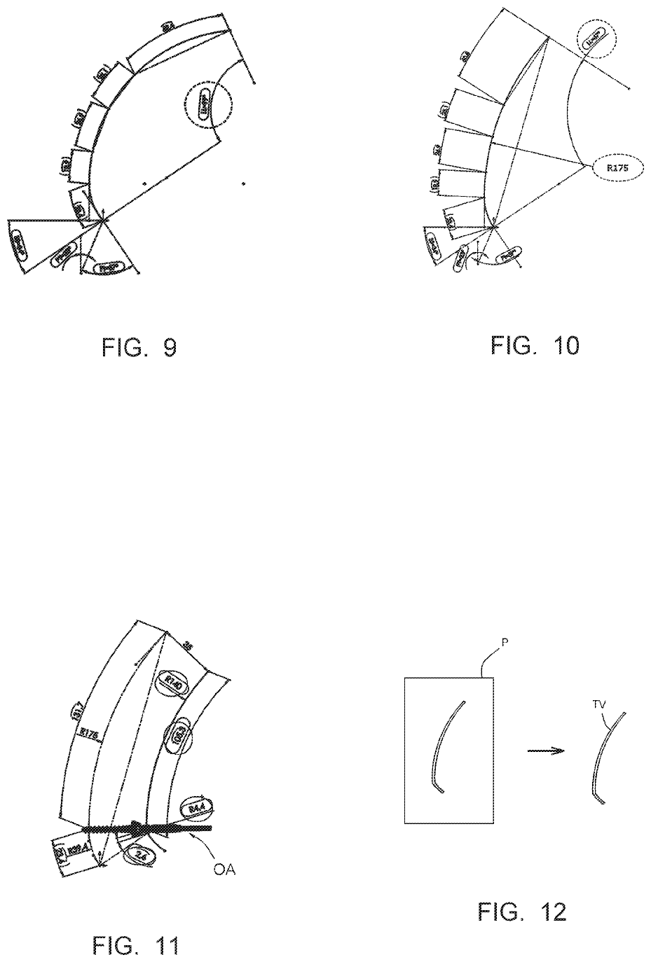

FIG. 9: a reference centered at the plate of S1 is defined from said pelvic parameters, the origin point of that reference being the central point of that plate, then the different points of said cloud of points, attached to each vertebra, are positioned in that reference, and arcs between the identified points are drawn step by step, all of the arcs being tangent to each other and the arc extending from S1 being tangent to the straight line perpendicular to the plate of S1;

FIG. 10: the values of the arc lengths are read, and the correction to be applied to the lumbar segment to be treated is simulated as follows:
drawing a straight line tangent to the desired postoperative apex point, moving that straight line to a vertical position such that the arc attached to that straight line is tangent to that desired postoperative apex point, the latter thus being repositioned so as to become the apex point of the modeled vertebral column segment,
defining, as co-radial to each other, the arcs situated below that apex point and defining, as co-radial to each other, the arcs situated above that same apex point, so as to obtain two different curvatures, one above that apex point and the other below that apex point,
defining the lumbar lordosis as being equal to plus or minus ten degrees of the pelvic incidence, and defining, as desired, one of the three following values:
   SVA distance smaller than 5 cm;
   SVA/SFD ratio comprised between −1.9 and +0.1; the value of this ratio is positive on the side of the vertical V1 situated toward the femoral heads TF and is negative on the side of that vertical V1 situated opposite the femoral heads TF;
   T1/SPI angle comprised between −9° and 0 0; this angle is negative on the side of the vertical V3 at the center of T1 located toward the femoral heads TF;
   defining two arcs concentric to the two curvatures obtained in the above step, which are tangent to each other at the apex point, those arcs forming a curved segment SC representing the ideal curvature of the rod to be implanted in order to obtain the correction of the vertebral segment to be treated.

FIG. 11: the curved segment SC is translated away from the mean line of the vertebral column, over an evaluated mean distance going from the center of the vertebrae to the anchoring points of the anchoring members OA (FIG. 10, it is a pedicle screw) for anchoring the rod to the vertebrae of said vertebral segment to be treated, such that the position of said curved segment SC corresponds to the position that the rod will assume once implanted; the diameter of the rod to be implanted being defined, a two- or three-dimensional model of that rod is then established, curved along said arcs.

FIG. 12: a drawing or plan P is established from that model, then the vertebral rod TV to be obtained is arranged from that drawing P, in particular by cold bending.

The method according to the invention thus has the decisive advantage of making it possible to produce the ideal curvature of a rod for vertebral osteosynthesis material designed to support a patient's vertebral column.

What is claimed is:

1. A system for producing a customized spinal rod to support a vertebral column of a patient, the system comprising:
an interface comprising a computer processor and an electronic storage medium, the interface configured to analyze a preoperative image of a vertebral column of a patient by:
identifying, on the preoperative image, points defining curvature of the vertebral column; and
deducing a morphotype to which the vertebral column corresponds based on the identified points;
the interface further configured to:
simulate a correction to be applied to a vertebral segment to be treated, wherein simulating the correction comprises one or more of:
repositioning an apex point comprising a forwardmost point of the vertebral column on the preoperative image; or
redefining one or more pelvic parameters;
deduce a curved segment representing a desired curvature of the customized spinal rod based at least in part on the simulated correction;
establish a drawing or plan of the customized spinal rod based on the deduced curved segment; and
cause a straight rod to be bent according to the drawing or plan.

2. The system of claim 1, wherein redefining one or more pelvic parameters comprises redefining lumbar lordosis to be within ten degrees of a pelvic incidence.

3. The system of claim 1, wherein the one or more pelvic parameters comprises one or more of a sacral slope, pelvic version, or pelvic incidence.

4. The system of claim 1, wherein the preoperative image comprises a sagittal x-ray image.

5. The system of claim 1, wherein the preoperative image comprises an image of cervical vertebrae.

6. The system of claim 1, wherein the customized spinal rod comprises vertebral osteosynthesis material.

7. The system of claim 1, wherein the preoperative image is further analyzed by identifying, on the preoperative image, one or more of:
a SVA distance from a vertical of a posterior upper point of a plate of a first vertebra of a sacrum (S1) to a vertical passing through a center of a seventh cervical vertebra;
a SFD distance from the vertical of the posterior upper point of the plate of S1 to a vertical passing through a center of a femoral head; or
a T1/SPI angle formed between a segment going from a center of a first dorsal vertebra (T1) to the center of the femoral head and a vertical line extending from the center of T1.

8. The system of claim 1, wherein the interface is further configured to generate a two-dimensional or three-dimensional model of the customized spinal rod.

9. The system of claim 1, wherein the interface is further configured to generate a wire model of the vertebral column.

10. The system of claim 1, wherein the interface is further configured to generate a desired diameter of the customized spinal rod.

11. The system of claim 1, wherein the interface further is configured to cause the straight rod to be bent according to the drawing or plan by transferring the drawing or plan to a service provider, causing a production unit to bend the straight rod to have the desired curvature.

12. A method for producing a desired curvature of a customized spinal rod to support a vertebral column of a patient, the method comprising:
identifying, on a preoperative image, points defining curvature of the vertebral column; and
deducing a morphotype to which the vertebral column corresponds based on the identified points;
simulating a correction to be applied to a vertebral segment to be treated, wherein simulating the correction comprises one or more of:
repositioning an apex point comprising a forwardmost point of the vertebral column on the preoperative image; or
redefining one or more pelvic parameters;
deducing a curved segment representing a desired curvature of the customized spinal rod based at least in part on the simulated correction;
establishing a drawing or plan of the customized spinal rod based on the deduced curved segment; and
causing a straight rod to be bent according to the drawing or plan.

13. The method of claim 12, further comprising generating a two-dimensional or three-dimensional model of the customized spinal rod.

14. The method of claim 12, further comprising generating a wire model of the vertebral column.

15. The method of claim 12, further comprising generating a desired diameter of the customized spinal rod.

16. The method of claim 12, wherein redefining the one or more pelvic parameters comprises redefining lumbar lordosis to be within ten degrees of a pelvic incidence.

17. The method of claim 12, wherein causing the straight rod to be bent according to the drawing or plan comprises transferring the drawing or plan to a service provider, causing a production unit to bend the straight rod to have the desired curvature.
